(19) United States
(12) Patent Application Publication
(10) Pub. No.: US 2022/0409826 A1
(43) Pub. Date: Dec. 29, 2022

(54) Systems and Methods for Automatic Intravenous Injections

(71) Applicants: Craig SKILLMAN, Princeton, NJ (US); Miles COLE, San Francisco, CA (US); The Trustees of Princeton University, Princeton, NJ (US); Invictis Labs, Inc., San Francisco, CA (US)

(72) Inventors: Craig B. Arnold, Skillman, NJ (US); Miles Cole, San Francisco, CA (US)

(73) Assignees: The Trustees of Princeton University, Princeton, NJ (US); Invictis Labs, Inc., San Francisco, CA (US)

(21) Appl. No.: 17/756,246

(22) PCT Filed: Nov. 19, 2020
(86) PCT No.: PCT/US2020/061375

§ 371 Date: May 19, 2022

Related U.S. Application Data
(60) Provisional application No. 62/937,578, filed on Nov. 19, 2019.

(51) Int. Cl. A61M 5/42; A61M 5/20; A61M 5/46; A61M 5/32

(52) U.S. Cl. CPC A61M 5/427; A61M 5/2053; A61M 5/46; A61M 5/3287; A61M 2005/206; A61M 2205/3306; A61M 2205/3303; A61M 2205/50

(57) ABSTRACT

Systems and methods for automatic intravenous injection in accordance with embodiments of the invention are illustrated. One embodiment includes a method for automatically injecting a needle into a vein. The method includes steps for identifying an injection position using a first set of one or more sensors, positioning an injection mechanism at the identified injection position, and vertically inserting a needle until entry in a vein is detected using a second set of one or more sensors.

### CROSS-REFERENCE TO RELATED APPLICATIONS

The current application claims the benefit of and priority under 35 U.S.C. § 119(e) to U.S. Provisional Patent Application No. 62/937,578 entitled “Automatic Intravenous Injection Device” filed Nov. 19, 2019. The disclosure of U.S. Provisional Patent Application No. 62/937,578 is hereby incorporated by reference in its entirety for all purposes.

### FIELD OF THE INVENTION

The present invention generally relates to injection devices and, more specifically, automatic intravenous injection devices.

### BACKGROUND

In the midst of an evolving technological world, venipuncture for the direct injection of medication and the drawing of blood has basically not changed since the practice was first developed in the 1830's despite the inefficiency, unreliability, and potential harm that can arise. Under the most ideal conditions it is difficult even for a trained nurse to access a vein consistently due to practical limitations of human motor skills, changing variables from person to person such as vein size, shape, strength, health, etc., as well as basic human error. Of course, under non-ideal conditions, the risks are even greater.

Perhaps the greatest risks are infection caused by large needle punctures, scarring, and other sharps related injuries to both the injectee and the person doing the injection. In fact, the Center for Disease Control and Prevention (CDC) estimates that an average of 1,000 sharps related injuries are sustained by hospital-based healthcare personnel per day nationwide (CDC, 2004). These needle stick injuries result in the transmission of bloodborne viruses and are estimated to cost the healthcare sector in the United States over $3 billion a year. Additionally, according to a new research report by Global Market Insights, bloodborne infections in American hospitals account for around 99,000 deaths per year. Such injuries and infections are 100% avoidable yet despite these challenges, millions of Americans require IV injections every day for a wide range of infusion therapy treatments and a large percentage of these can require the person themselves or another, untrained assistant to perform the venipuncture.

Important spaces for automatic detection and insertion include hemophilia, blood draws, and chemotherapy. Hemophiliacs require daily IV injections, and either a trained nurse must come to a patient's house, the patient must travel to a treatment center, or the patient must do the injection themselves. Even in a hospital setting, around 25% of first stick IV infusion attempts are unsuccessful, illustrating the difficulty of injecting a vein in small or weakened patients. Hemophiliacs, cancer patients, and patients with diseases treated by IV infusions must go through the mentally and physically taxing process of learning to self-infuse as does their family, especially in the case of children. As such, an easy-to-use system capable of providing accurate intravenous injection while removing the risk of human error is useful and desirable.

### SUMMARY OF THE INVENTION

Systems and methods for automatic intravenous injection in accordance with embodiments of the invention are illustrated. One embodiment includes a method for automatically injecting a needle into a vein. The method includes steps for identifying an injection position using a first set of one or more sensors, positioning an injection mechanism at the identified injection position, and vertically inserting a needle until entry in a vein is detected using a second set of one or more sensors.

In a further embodiment, the first set of sensors includes at least one of the group consisting of an infrared sensor, an optical sensor, and ultrasound.

In still another embodiment, positioning the injection mechanism includes moving the injection mechanism along a first axis and vertically inserting the needle includes moving the injection mechanism along a second perpendicular axis.

In a still further embodiment, the method further includes steps for securing a depth of the injection mechanism when entry in the vein is detected.

In yet another embodiment, the needle is part of a needle element, the needle element includes a needle housing, and securing the depth of the injection mechanism includes locking a position of the needle housing.

In a yet further embodiment, the method further includes steps for positioning a set of one or more vein roll prevention bars around the injection position.

In another additional embodiment, the second set of sensors includes at least one of the group consisting of a force sensor, an electrochemical impedance sensor,

In a further additional embodiment, the second set of sensors includes first and second electrochemical impedance sensors, wherein detecting entry in the vein includes detecting the presence of a liquid at both the first and second electrochemical impedance sensors.

In another embodiment again, a set of lumen holes of the needle are located between the first and second electrochemical impedance sensors.

In a further embodiment again, the method further includes steps for creating a pressurized space around the injection position.

In still yet another embodiment, the method further includes steps for sterilizing an area around the injection position.

In a still yet further embodiment, vertically inserting a needle comprises vertically advancing a needle into a user, causing a needle housing surrounding the needle to retract, and when entry in the vein is detected, locking the needle housing so that it cannot retract further.

In still another additional embodiment, vertically inserting a needle includes rotating the needle around an axis of the needle.

In a still further additional embodiment, vertically inserting a needle includes oscillating the needle.

In still another embodiment again, detecting entry in the vein includes determining a force measurement based on an oscillation frequency of the oscillation.

In a still further embodiment again, the method further includes steps for generating a 2-D map using the first set of sensors and identifying the injection position based on the generated 2-D map. 17. An injection system for automatically injecting a needle, the injection system comprising a housing, an injection mechanism comprising a securing element for securing a needle element, a positioning element for positioning the needle element along a first axis, and an insertion element for inserting the needle element along a perpendicular second axis, a set of one or more processors configured to identify an injection position using a first set of one or more sensors, position an injection mechanism at the identified injection position along the first axis, and vertically insert a needle along the second axis until entry in a vein is detected using a second set of one or more sensors.

In yet another additional embodiment, the housing includes an injection opening and at least two roll prevention bars placed in parallel along the injection opening.

In a yet further additional embodiment, the needle element includes a coupling assembly for connecting the needle to the injection mechanism and the needle.

In yet another embodiment again, the coupling assembly provides a connection for liquids passing through the needle, wherein the connection is perpendicular to the needle.

In a yet further embodiment again, the needle element further includes a needle housing that surrounds the needle.

In another additional embodiment again, the needle housing includes a locking mechanism, wherein the set of processors is further configured to lock the locking mechanism when a vein is detected.

In a further additional embodiment again, the needle housing includes a sheath that covers an opening of the needle housing.

In still yet another additional embodiment, the needle housing is a retractable needle housing.

In a further embodiment, the needle includes a first electrochemical sensor below a lumen opening of the needle and a second electrochemical sensor above the lumen opening.

In still another embodiment, the needle tip is a non-linear tip.

Disclosed is a system for automatically injecting a needle into a user's vein in a direction perpendicular (or normal) to the surface of the user's skin. In certain embodiments, automatic injection systems can include a clamp mount for securing a needle, sensors to detect and identify the position of a vein, an injection mechanism to maneuver the clamp mount in a direction parallel to the surface of the user's skin and to inject the needle into a vein in a direction perpendicular to the surface of the user's skin, a sensor to determine when the needle has pierced the vein, and a processor to control the system.

Vein identification sensors in accordance with a number of embodiments of the invention may be based on optical inspection, ultrasound, thermal imaging, and/or proximity sensors. The motors may be linear or piezoelectric motors. The system may include a tube connecting the needle to a syringe. The needles may be a 32-gauge 4 mm needle, a 27-gauge 12.7 mm needle, a 26-gauge 12.7 mm needle, or a 25-gauge 15.9 mm needle. Systems in accordance with many embodiments of the invention may also include a vein identifying light, a thermal imaging sensor for verifying the injection point is suitable for injection, and/or a vein roll prevention bar.

In numerous embodiments, automatic injection systems may also advantageously include a cuff, where the clamp mount, sensors, and motors are contained on or within the cuff, and wherein the cuff is capable of being in direct contact with a patient's body. The cuff may be designed to fit on an arm or a leg. The system may contain a battery and may contain an electrical connector to recharge the battery. The system may contain a wireless transmitter. The system may contain one or more buttons, such as (but not limited to) a start button, stop button, and/or an eject button.

The system disclosed herein solves the problems associated with human error during intravenous injections. By precisely and automatically injecting a needle into a vein, the disclosed system will prove to be very cost efficient for hospitals, insurance companies, the federal government, and private users alike.

As used herein, “vertical” or “vertically” is intended to mean a direction perpendicular or normal to the surface of the user's skin, regardless of the actual absolute orientation of the system. In a variety of embodiments, vertical may also refer to a direction perpendicular to a positioning plane, where a needle is positioned along the plane and the injection occurs perpendicularly to the plane.

As used herein, “lateral” or “laterally” is intended to mean a direction parallel to the surface of the user's skin, regardless of the actual absolute orientation of the system.

As used herein, “substantially vertical” or “substantially lateral” is intended to mean a direction that is normal or parallel to the user's skin, respectively, ±5 degrees. That is, it is intended to cover those situations where the direction is not perfectly vertical, or not perfectly parallel, but a skilled artisan would understand the direction to be generally normal or parallel to the user's skin, respectively.

As used herein, “system” is intended to cover both a singular device, and a system where components are spread across multiple devices. While a singular device is a preferred embodiment, it is recognized that with software, it is possible to offload some processing power to a remote source, or to provide a remote user with control over the software or functioning of the device, with relative ease, and thus the disclosed apparatus may actually utilize multiple devices.

The description and claims will be more fully understood with reference to the following figures and data graphs, which are presented as exemplary embodiments of the invention and should not be construed as a complete recitation of the scope of the invention.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates an example of an automatic injection device in accordance with an embodiment of the invention.

FIG. 2 illustrates an example of an injection opening for an automatic injection device in accordance with an embodiment of the invention.

FIG. 3 illustrates an exposed view of an automatic injection device in accordance with an embodiment of the invention.

FIG. 4 illustrates an example of a needle element in accordance with an embodiment of the invention.

FIG. 5 illustrates an example of a needle element with a needle housing in accordance with an embodiment of the invention.

FIG. 6 illustrates an example of a needle housing in accordance with an embodiment of the invention.

FIG. 7 illustrates an example of a retractable needle housing in accordance with an embodiment of the invention.

FIG. 8 illustrates examples of needle tips in accordance with some embodiments of the invention.

FIG. 9 illustrates examples of lumen openings in accordance with a number of embodiments of the invention.

FIG. 10 conceptually illustrates a process for automatic injections in accordance with an embodiment of the invention.

FIG. 11 illustrates an example of an automatic injection system that automatically injects in accordance with an embodiment of the invention.

FIG. 12 illustrates an example of an automatic injection element that executes instructions to perform processes that automatically inject a needle in accordance with an embodiment of the invention.

### DETAILED DESCRIPTION

### Automatic Intravenous Injection System

Systems and methods in accordance with numerous embodiments of the invention for automatic intravenous injection are disclosed. Automatic injection systems in accordance with a variety of embodiments of the invention can be portable medical systems that can automatically sense the location of a vein and inject a needle to precisely pierce the vein without damaging surrounding tissue. In many embodiments, automatic injection systems can be operated by an individual without any additional assistance. Automatic injection systems in accordance with a number of embodiments of the invention can employ a portable arm cuff style. In several embodiments, automatic injection systems can be incorporated as part of a fixed stationary cuff system, where a patient's arm may be placed in the device, rather than affixing a device to the patient. Automatic injection systems in accordance with many embodiments of the invention can be used, inter alia, in hospital, military, blood draw centers, home infusion, infusion treatment center, veterinary, emergency response, school, or disaster relief settings.

Systems in accordance with a number of embodiments of the invention can be used to inject a needle into a vein for the purpose of infusing medication intravenously. In a number of embodiments, automatic injection systems can be used for various other purposes, such as (but not limited to) drawing blood (e.g., using a system configured to allow blood collection tubes to be inserted and removed), dialysis (e.g., using two systems, one for drawing blood for a dialysis machine, and one for returning the blood to the subject), tissue biopsy (e.g., using an appropriate needle for the biopsy), and/or other medical procedures. Automatic injection systems in accordance with several embodiments of the invention can be used with non-human subjects, such as in certain veterinarian applications.

Systems and methods in accordance with many embodiments of the invention can employ vertical injection technology (VIT). VIT can inject a needle at a near 90-degree angle from the surface of the skin, in contrast to the typical 45-degree (or other) angle that a doctor or nurse might use. With many other existing solutions, a user would be forced to position an arm in a very specific, and many times, uncomfortable position. However, as systems in accordance with a variety of embodiments of the invention can be used in various orientations, users can be in a more relaxed and comfortable position.

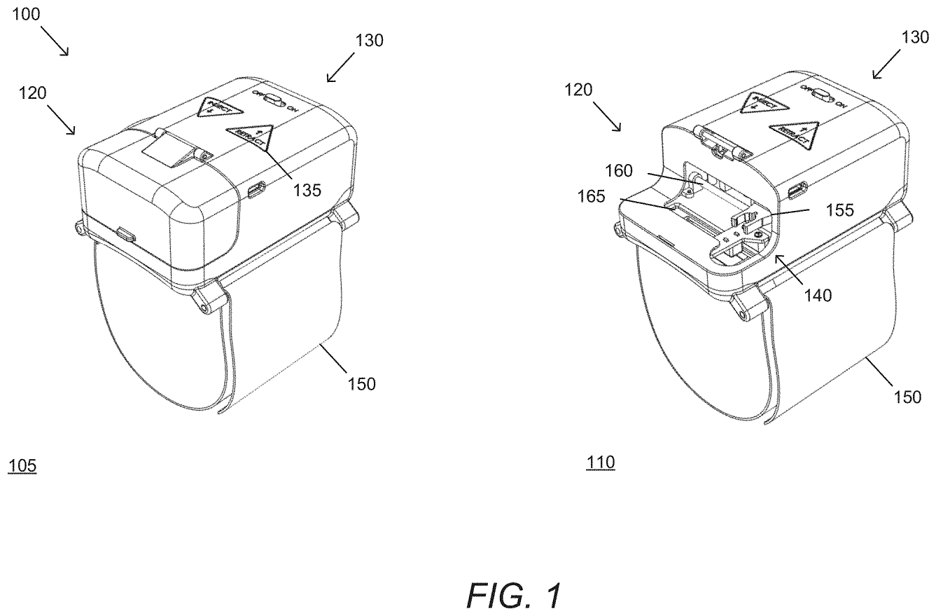

An example of an automatic injection device in accordance with an embodiment of the invention is illustrated in FIG. 1. This figure illustrates closed and open views 105 and 110 of an automatic injection element 100. Closed view 105 illustrates automatic injection element 100 with housing 110 and a cuff 140. Housing 110 is attached to cuff 150. Housing 110 includes controls 115 and a covered injection compartment 120. Open view 110 illustrates housing 110 with an uncovered injection compartment 120, which includes an injection mechanism 140. In this example, clamp mount 155 moved along track bar 160, positioning a needle element along a single axis before injection through injection opening 165.

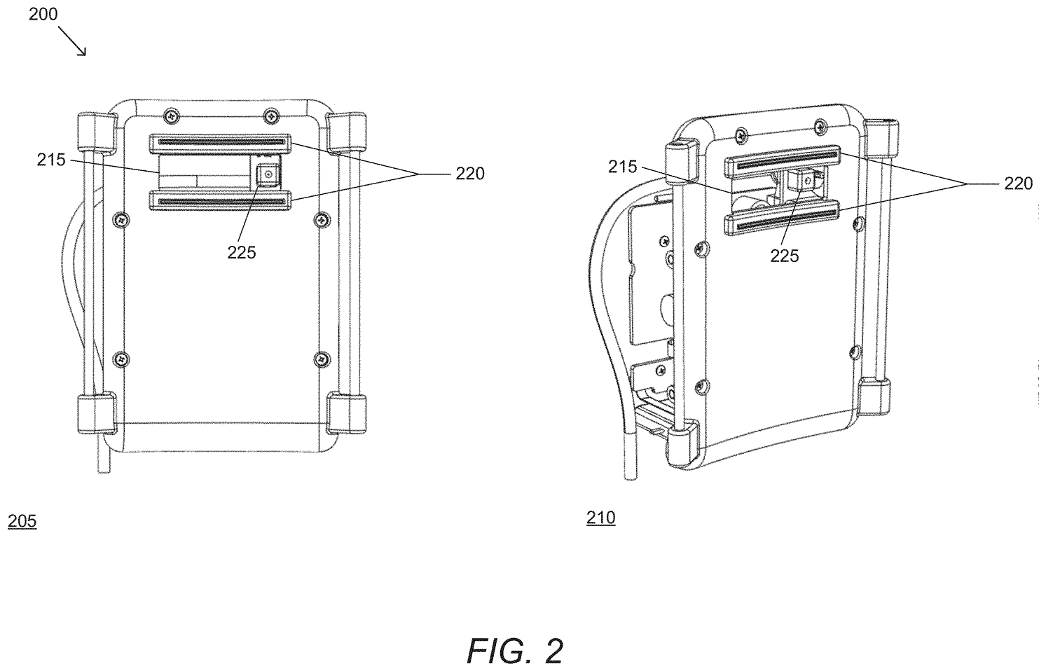

An example of an injection opening for an automatic injection device in accordance with an embodiment of the invention is illustrated in two views 205 and 210 of FIG. 2. In these views, the bottom of an automatic injection device which may be in contact with a user's skin is illustrated. The bottom includes an injection opening 215 and vein roll prevention bars 220. A bottom view of a needle element 225 is also visible through injection opening 215.

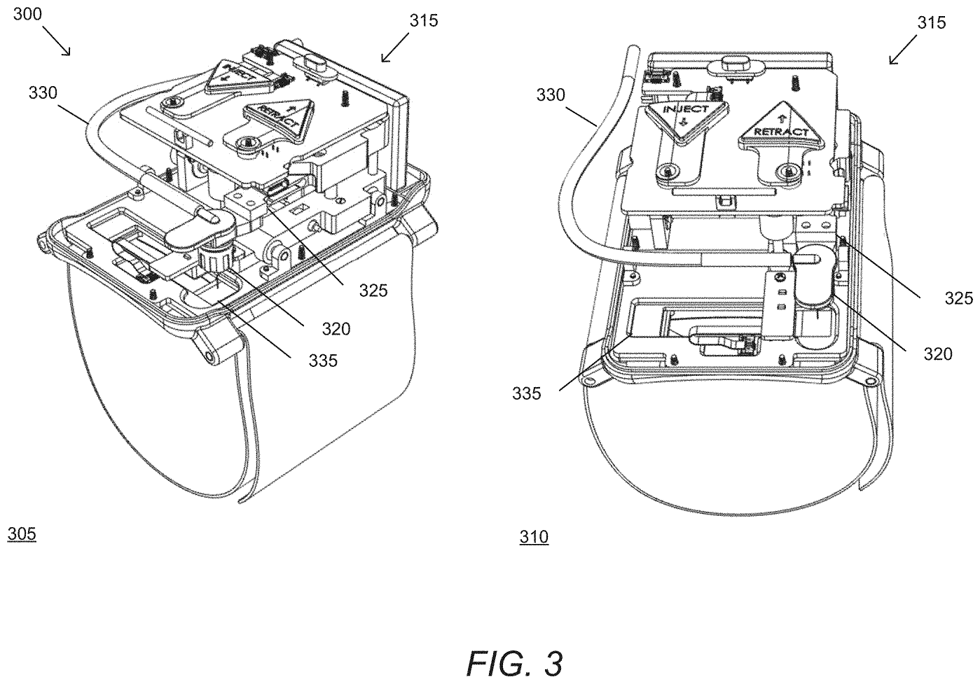

An exposed view of an automatic injection device in accordance with an embodiment of the invention is illustrated in two views 305 and 310 of FIG. 3. In particular, the exposed views 305 and 310 illustrate various electronic circuitry 315 and needle element 320 inserted into clamp mount 325. Needle element 320 includes a tube 330 for injecting into and/or drawing from a vein. Clamp mount 325 secures needle element 320 and motors and the electronic circuitry 315 can be used to position the needle element over injection opening 335.

Although a specific example of automatic injection devices are illustrated in FIGS. 1-3, any of a variety of configurations (e.g., with more or fewer components, various arrangements, etc.) of automatic injection devices can be utilized to perform processes for automatic injection similar to those described herein as appropriate to the requirements of specific applications in accordance with embodiments of the invention. More detailed descriptions of various components of automatic injection systems in accordance with a variety of embodiments of the invention are described in greater detail below.

### Housing

Housings in accordance with several embodiments of the invention can enclose various internal components, such as (but not limited to) electric circuitry that can be used to control an automatic injection device. In several embodiments, housing can be an enclosure (e.g., plastic, ceramic, and/or metal) that prevents access to the electric circuitry and motors, while still providing an injection opening for accessing an injection site, as well as allowing a user to have access to an injection compartment in order to install or replace the needle element.

Housings in accordance with several embodiments of the invention can include various elements, such as (but not limited to) controls, injection compartments, and/or injection openings. Controls can be used to perform various operations with an automatic injection device, such as (but not limited to) inserting a new needle element, ejecting a used needle element, performing an injection, powering on/off the device, sterilizing an injection site, etc. Housings in accordance with some embodiments of the invention can contain one or more ports (not shown), such as a port for accepting an electric power connection.

In some embodiments, housings can include injection compartments to enclose the injection site and protect it from outside contaminants (e.g., dust, viruses, bacteria, etc.). This can be particularly important when the injected needle is maintained for longer periods of time (e.g., with IV injections, dialysis, etc.). Injection compartments in accordance with various embodiments of the invention can provide a user access to insert needle elements into the device. In several embodiments, injection compartments can be used to sterilize and/or pressurize an injection site to facilitate the injection process. Injection compartments in accordance with certain embodiments of the invention can include sterilization components (e.g., ultraviolet, sterilization solutions, disinfectant sprays, etc.) for sterilizing an injection site prior to injection. In some embodiments, injection compartments can be pressurized to create a vacuum so no contaminants can leak through the space between the patient's arm and the device. Pressurized injection compartments in accordance with various embodiments of the invention can reduce movement of an injection device at the injection site. In some embodiments, injection compartments can be sealed to prevent outside elements from entering during an injection process.

In a variety of embodiments, housings can include injection openings through which injections can be performed. Vein roll prevention bars in accordance with a number of embodiments of the invention may be located at edges of an injection opening to reduce vein movement during an injection process. In a variety of embodiments, roll prevention bars can be parallel protrusions extending from the bottom of an injection device that come in contact with a patient's skin when an automatic injection system is placed on the patient and can help to hold the vein steady during an injection, increasing the likelihood of successfully entering a vein. In many embodiments, roll prevention bars can include a pair of bars that run perpendicular to the targeted vein in order to cross the vein in two locations. In several embodiments, the distance between a pair of roll bars can be wide enough to allow the needle and/or needle housing to interact with the skin, but not so large as to provide no benefit to the anti-roll objective. In a variety of embodiments, the width of the roll prevention bars can be as long or longer as the lateral translation of the needle assembly provided by the device. Since the body surface may have a curvature (e.g., on an arm or leg) that is different than the curvature of the roll prevention bars, vein roll prevention bars in accordance with many embodiments of the invention can be minimally sized to reduce discomfort and device size. In certain embodiments, roll prevention bars can be adjustable. After a vein has been identified, automatic injection devices in accordance with some embodiments of the invention can lower and/or position roll prevention bar(s) to prevent the target vein from rolling prior to performing an injection.

Vein roll prevention bars in accordance with several embodiments of the invention can include lights and/or sensors that can be used to assist in locating veins. In a variety of embodiments, lights from the vein roll bars can light up a region of a user's skin to facilitate the detection of veins in the region. The benefit of integrating the light into the roll bars is that it can optimally couple the light into the skin, reducing the occurrence of stray reflectance that can blind or saturate an image sensor. Vein roll prevention bars in accordance with a variety of embodiments of the invention can be constructed of optically transparent or transmissive material. This can allow IR and/or red light to be transmitted through the roll prevention bars into the skin. In some embodiments, this light can be reflected back and detected by a vein detection system in order to locate the position of veins in the patient.

In some embodiments, housings can be removably attached to a cuff. Cuffs in accordance with a variety of embodiments of the invention can be configured to wrap around the outer surface of a subject's body, such as (but not limited to) an arm or leg. In many embodiments, cuffs may only partially wrap around an extremity. In several embodiments, cuffs can also help to prevent movement of the housing while in operation. Cuffs in accordance with a variety of embodiments of the invention can be made of various materials, such as (but not limited to) plastic, rubber, and/or neoprene. In many embodiments, cuffs can be any appropriate flexible substrate. In some embodiments, cuffs can be removable from housing and/or disposable. Cuffs in accordance with certain embodiments of the invention can utilize material that can be repeatedly sterilized. In some embodiments, cuffs include a woven or nonwoven material. Cuffs in accordance with a variety of embodiments of the invention can be comprised of a natural or synthetic material, including (but not limited to) cotton, wool, polypropylene, polyethylene, silicone, styrenic block copolymers, etc. In some embodiments, cuffs can comprise a non-woven material. Cuffs in accordance with a number of embodiments of the invention can include a stretchable material.

In several embodiments, cuffs can be wrapped around an arm or leg, similar to how an upper arm blood pressure cuff operates. In a number of embodiments, no cuff is present, or the cuff is merely a relatively small substrate under the housing. In some embodiments, a user can simply place, or adhere, the housing into place on an arm, leg, etc. Cuffs in accordance with a number of embodiments of the invention may also comprise, e.g., extensible fabric, hook-and-loop fasteners, buttons, and/or adhesive.

In some embodiments, cuffs can be inflatable. For example, the cuff may have a hard, outer shell with rubber inflatable tubing on the inside, large enough to encompass arms or legs of, e.g., a typical human being. The rubber tubing may remain deflated while the cuff is slid into position. Once an automatic injection system is in position, tubing can then be automatically or manually inflated until system is tightly secured in place. This tubing's constriction on the arm may also be configured to act as a tourniquet and can also prevent the target vein from rolling. In some embodiments, an inflation pump can be used, that works by squeezing. The pump is operably connected to the tube, and by squeezing multiple times, air is pushed into the tubing to make the cuff tighter on the user's appendage.

In numerous embodiments, cuffs may include various components, such as sensors and/or lights that can be used for various purposes, such as (but not limited to) vein detection, vein entry detection, needle tracking, blood pressure monitoring, pulse monitoring, oxygenation level monitoring, glucose monitoring, etc. In numerous embodiments, such information can be used to assist in various parts of the injection process. Processes in accordance with many embodiments of the invention can use a measure of the blood pressure to inform the force measurement, where higher blood pressure may indicate a particular force response to a needle entering a vein as compared to a lower blood pressure. In a variety of embodiments, oxygenation levels of the blood can be used to supplement an imaging system to help identify the location of the veins.

Although many of the examples described herein describe a portable cuffed injection device, one skilled in the art will recognize that similar systems and methods can be used in a variety of applications, including (but not limited to) stationary injection devices and/or cuffless devices, without departing from this invention.

### Injection Mechanism

Automatic injection devices in accordance with a number of embodiments of the invention can use an injection mechanism to position a needle element over an identified injection site and inject a needle into a vein. In various embodiments, injection mechanisms can include securing mechanisms for securing a needle element, positioning elements for positioning the needle element over an injection site, and/or injection elements for inserting a needle of the needle element into a vein.

Securing mechanisms in accordance with numerous embodiments of the invention can include any of a variety of fasteners for securing a needle element, such as (but not limited to) clamp mounts, clips, screws, bolts, adhesive, hook and loop tape, snap fit, friction fit, magnets, etc. Securing mechanisms in accordance with several embodiments of the invention can be used to securely connect with needle elements. In some embodiments, securing mechanisms can allow a needle to be threaded onto it, such as (but not limited to) via a LUER-LOCK or other standardized threading mechanism. Securing mechanisms in accordance with several embodiments of the invention can allow needles to be pressure fit into it.

In some embodiments, securing mechanisms can be configured to receive and secure commercially available needles, such as 32-gauge 4 mm needles, 27-gauge 12.7 mm needles, 26-gauge 12.7 mm needles, and/or 25-gauge 15.9 mm needles. In certain embodiments, securing mechanisms can be configured to secure a syringe. Needles in accordance with numerous embodiments of the invention can be part of a custom needle element. Needle elements in accordance with various embodiments of the invention are described in greater detail below.

Securing mechanisms in accordance with certain embodiments of the invention can be operably connected to one or more positioning elements (e.g., motors, electronic circuitry, track bars, etc.) that control the movement of the needle in a lateral direction above the user's skin. In some embodiments, positioning elements are configured to move along a single axis (e.g., across a user's arm). Alternatively, positioning elements in accordance with numerous embodiments of the invention can move along two axes (e.g., up/down and across a user's arm). In various embodiments, positioning elements can be configured to move the securing mechanism in and out of the housing, so as to allow a user easier access to the securing mechanism and/or needle. In various embodiments, injection elements (e.g., motors, electronic circuitry, etc.) can be used to securely insert a needle through a user's skin into a vein. Injection elements in accordance with some embodiments of the invention can include motors that control the movement of the needle in a vertical direction.

Motors in accordance with several embodiments of the invention may utilize various types of motors, such as (but not limited to) linear motors, piezoelectric motors, worm drives, gears, and/or belts, or other appropriate techniques as understood by a skilled artisan. In some embodiments, electronic circuitry can include one or more processors, one or more sensors (e.g., vein detection and/or position sensors, vein insertion detection sensors, and/or thermal imaging sensors). In some embodiments, processors can be configured (e.g., via a set of instructions) to process information from sensors as part of a control system, allowing automatic injection systems to perform various operations, such as (but not limited to) detecting a vein, positioning a needle correctly for injection into that vein, detecting when the needle has entered a vein, and/or determining if a given vein is appropriate for injection. Electric circuitry in accordance with some embodiments of the invention may also include various other elements including, but not limited to, a power source, power connection, indicators, displays, and/or buttons. In some embodiments, one or more of these internal components may be in communication with the one or more processors.

### Control System

Control systems in accordance with certain embodiments of the invention can be used to identify the position of a vein, position a needle element over the identified position, inject the needle, determine whether the vein has been punctured, perform intravenous operations, and/or withdraw the needle. In a variety of embodiments, vertical injections can allow a control system to operate on only two axes. As each injection is at the same 90 degree angle, control systems in accordance with various embodiments of the invention can position needle elements over a vein along a single axis (e.g., scanning horizontally over the veins of the arm), and inject the needle along another axis that moves the needle vertically down on the z plane to inject into the vein. This can allow for simpler, smaller, and more efficient injection systems.

In many embodiments, control systems can detect vein locations and/or the puncture of a vein by a needle to drive an injection process. Control systems in accordance with certain embodiments of the invention can use a set of one or more components (e.g., sensors, lights, transmitters, receivers, etc.) to detect vein locations and/or the puncture of a vein by a needle. Examples of sensors in accordance with certain embodiments of the invention are described in greater detail below.

In a number of embodiments, control systems can be configured to rotate needle elements during an injection. Rotation motions can be useful when a needle tip has a screw-type design. In this case, rather than a linear z axis, control systems in accordance with certain embodiments of the invention can include a rotating motor and allow the position to move based on the pitch of the screw. In a number of embodiments, it can be beneficial to include linear motion as well in order to ensure the proper force is being applied to the system. This would create a third motion control axis without significantly increasing the size of an injection device.

Control systems in accordance with a variety of embodiments of the invention can be configured to oscillate (or vibrate) of needle elements during an injection. Oscillation on the z-axis (injection axis) can provide noticeable and important benefits as compared to simply linear motion. Oscillatory motion in accordance with a number of embodiments of the invention is the combinate of linear motion with a sinusoidal oscillation in motion. As a result, the mean displacement of the needle will continue to increase, while the actual motion will oscillate about this mean value.

This type of motion can have many benefits for the injection of the needle. Oscillating needles in accordance with numerous embodiments of the invention, particularly with a rough (or textured) surface, may experience a lower friction as compared to a linearly moving needle. As a result, the injection can be performed more accurately and with less potential damage/discomfort to the patient. In a number of embodiments, oscillation can also allow for noise reduction in force measurements (e.g., for vein penetration detection).

In constructing an oscillatory motion either to benefit the force measurement or the motion of the needle, it is determined that ultrasonic frequencies are optimal (frequencies greater than 20 kHz). At frequencies below 20 kHz, there is the potential for auditory discomfort to a person or other vibration induced discomforts (e.g. nausea, dizziness, tinitis, etc.). If the frequency is too high, then one has to be concerned with RF disruption to communications systems such as BLUETOOTH or WI-FI, or other RF sensitive devices that might be present in or around a patient (e.g. pacemaker). In various embodiments, injection processes can oscillate at frequencies in the range of 20-100 kHz.

In numerous embodiments, the amplitude of the oscillation can vary depending on whether one is trying to optimize the force or the injection. Amplitudes that are too high (e.g. the range around the mean displacement) could lead to undesirable damage/pain to the patient. In the case of injection, the maximum amplitude of oscillation in accordance with a variety of embodiments of the invention can be smaller than the width of a vein and preferably smaller than half the width of the vein. In the case of force measurements, the maximum amplitude can be smaller than the thickness of the vein wall in accordance with a variety of embodiments of the invention. If the oscillation is greater than that, it may unintentionally pierce through the wall of the vein prematurely.

In a variety of embodiments, vein puncture detection can be performed using force sensing, where the oscillations of the needle can be used to reduce the noise in the force measurements. When measuring force with a fixed or linearly moving needle, there will be contributions of noise due to various sources such as the pulsing of blood, tremors/motions of the patients, and other mechanical noise (e.g. motors, etc.). However, by applying a known frequency of oscillation to the needle, vein puncture detection in accordance with various embodiments of the invention can restrict force measurements to analyze only those measurements that match the desired frequency. This type of lock-in approach would for instance allow the process to distinguish the difference and measure the pulse from the resistance force of the needle injection. In order to restrict the frequency analysis, one can take a Fourier transform of the force measurement (or Fast Fourier transform) and view the amplitude of the signal at the desired frequency as a function of time while the needle is being injected. As such, any change in force caused by for instance a pulse would be ignored by our measurement.

### Sensors

### Vein Position Detection Sensor

Vein position detection in accordance with a number of embodiments of the invention can include (but are not limited to) image sensors, light (e.g., visual light, IR, etc.) sensors, ultrasound sensors, thermal imaging, force sensors, proximity sensors, etc. In a variety of embodiments, control systems can use visual light cameras to capture images of the surface of the skin below the needle and/or light sources to generate light for the camera to be able to capture the image. Light sources in accordance with certain embodiments of the invention can pass light through the needle, can be offset from the needle, and/or can be applied directly to the skin (e.g., through the vein roll bars). In a number of embodiments, light sources can be attached to an outside wall of the housing, looking down towards the skin. In this fashion, it could be used to help the user identify a good target vein. The user could shine this light on their arm, and then place the cuff as close as possible to a potential vein made visible by this light.

In numerous embodiments, processes can use filters and image processing techniques to, e.g., identify a vein by determining a different colored vein-like portion of the image, and identify the edges of those veins. Processes in accordance with certain embodiments of the invention can determine a distance and direction the needle must move in order for the needle to be approximately centered within a detected vein, and can move the needle laterally until the needle is in the correct location. This process may be performed iteratively until the system verifies the needle is in the correct location. If no vein is detected, processes in accordance with certain embodiments of the invention can provide a notification (e.g., a sound, light, visual display, etc.) to indicate no vein had been detected, and that the cuff may need to be repositioned.

In a number of embodiments, infrared optical technology can be used to identify the location of a vein. This technology has been proven accurate at creating high resolution 2-D maps of veins and can be easily implemented by one of skill in the art with diode illumination and standard off-the-shelf-detectors. For example, control systems in accordance with a number of embodiments of the invention may irradiate an area with an infrared light to allow vein detection and positioning sensor(s) to capture an image showing the differential absorption and reflection of the light by subcutaneous veins and surrounding tissue. However, a skilled artisan will recognize that these approaches are currently known to be somewhat limited in their ability to measure depth. Thus, the vein detection and positioning sensor(s) can be used simply to identify a 2-D location of a vein under the outer surface of a subject's skin.

In many embodiments, infrared optical technology can be used with a laser or with a camera or ultrasound to determine when to stop the injection process. With a laser, IR lights shine on skin to make veins look like dark black lines and a barcode scanner-type laser can scan over the top and stop when it detects one of these black lines in accordance with many embodiments of the invention. With a camera, IR lights can shine on skin to make veins look like dark black lines and a camera can take a photo. In various embodiments, the photo can then be processed with image recognition to find the biggest black line in the photo and the needle can be positioned above this biggest black line. With ultrasound, an ultrasonic transducer can be applied to the patient's skin to send sound waves downwards, creating a map of all the veins and tissues based upon how these waves rebound. In various embodiments, a 3D image/render/mockup of all of the veins present can then be created from the ultrasound results and the needle can be positioned above where the best vein is.

In another embodiment, ultrasonic transducers or sensors are used to generate a series of images indicating blood flowing under a person's skin. The images can be processed using known image processing techniques, to identify the edges of where blood is flowing, thereby identifying a vein location.

Vein position detection sensors in accordance with various embodiments of the invention can include thermal imaging sensors. In a variety of embodiments, thermal imaging sensors can include a small infrared imaging system used to verify that the vein is suitable for injection by assessing that it has a healthy blood flow by distinguishing the vein temperature difference compared to the rest of the arm. In some embodiments, thermal imaging sensors can be positioned directly above the needle, looking vertically down onto a target vein insertion site area. In operation, thermal imaging sensors may use thermal infrared imaging to assess the difference in temperature on the user's forearm in order to roughly estimate the location of the vein. This can be used to distinguish a suitable vein from, e.g., a freckle. Once this rough estimation of the location of the vein is established, systems in accordance with numerous embodiments of the invention can send this information to other sensors to supplement a search to determine a more exact estimation of the vein location.

### Vein Insertion Detection Sensor

Vein insertion detection sensors in accordance with many embodiments of the invention can be utilized to determine when a needle has pierced a vein or otherwise been inserted into the vein. In several embodiments, vein insertion detection sensors can include (but are not limited to) force sensors, light sensors, ultrasound, and/or impedance sensors.

In numerous embodiments, vein insertion detection sensors can include a force sensor, such as a load cell, strain gage, or force-sensing resistor. In some embodiments, as a needle is vertically lowered towards a target insertion site in a vein, the resistance of the needle as it enters and passes through various features (skin, fat, muscle, vein, etc.) can be measured by vein insertion detection sensors. In some embodiments, the needle can be moved at a constant rate while the system measures the force and the displacement of the needle to determine the time the needle pierces the vein.

In some embodiments, sensors may be part of the securing mechanism, separate from the needle element. For example, as the needle is lowered, force sensors in accordance with several embodiments of the invention can detect the difference in friction between the needle pressing through skin tissue versus when the needle enters into the liquid (blood) of the vein. When a vein insertion detection process determines a difference in resistance that suggests that the needle has entered the vein, the control system in accordance with a variety of embodiments of the invention can send a signal to the stop the downward movement of the needle. In a variety of embodiments, the differences in resistance can be calibrated for intact skin, scar tissue, fat, muscle, etc., based on the particular needle being utilized. In some cases, automatic injection systems can include memory with calibration tables stored for each type of needle.

In a number of embodiments, vein insertion detection sensors can have a frequency 500 ms, 400 ms, 300 ms, 200 ms, or 100 ms. To determine whether the needle has entered a vein, vein insertion detection processes in accordance with numerous embodiments of the invention can consider the change in force from one measurement to the next and/or the absolute value of a measured force. If the measured values are within a predetermined range, processes in accordance with certain embodiments of the invention can determine that a vein has been entered. In some embodiments, trends in the measured values are tracked over a period of time (e.g., the time since a vein was detected or the time since a particular force threshold was met) in order to determine if a vein has been entered. For example, if the period of time being considered is the entirety of the injection, then during an injection, the needle must first pierce the dermis, then move through the epidermis, enter the subcutaneous tissue, and finally enter a vein. In this example, each of those different transition points may have a range for the change in force associated with the transition, and if the processor does not detect a change in force that matches the expected ranges of all the expected transitions, it may not make a determination that a vein has been entered.

In a number of embodiments, force measurements can allow an automatic injection system to determine if a vein is even hit, or if the vein has collapsed (rendering the injection impossible). For example, if the force sensor does not detect a force measurement within a given range (or no change in force measurement within a target range) that would indicate a vein has been entered, and the linear motor has travelled at least a particular distance (such as at least 10 mm, at least 5 mm, at least 4 mm, or at least 3 mm), the system can determine that the injection attempt has failed. In such cases, automatic injection systems in accordance with many embodiments of the invention can stop lowering the needle, and will raise the needle. In some embodiments, automatic injection systems can provide a notification (e.g., a sound, light, or visual display) to indicate that an injection attempt failed. In some embodiments, the system can attempt to reposition and enter the same vein. Automatic injection systems in accordance with many embodiments of the invention can attempt to locate a different vein and enter the different vein.

In certain embodiments, force measurements can be measured for an oscillating needle. By moving the needle at a proscribed oscillation frequency and amplitude during movement into the vein, one can use more advanced frequency analysis to improve the force measurement accuracy and more precisely determine when the vein is pierced. Such oscillations would be of an optimal frequency with a small amplitude so as not to cause pain to the patient.

Once a needle has been inserted into a vein, an intravenous operation (e.g., an infusion, blood draw, etc.) can be performed for the subject, through the needle. In a number of embodiments, for the entirety of the operation, vein insertion detection sensors can detect and adapt in real time to the changing position of the vein. For example, if the vein rises up a millimeter, the force sensors can recognize this and send a signal to the motors to also move the needle up a millimeter.

In other embodiments, automatic injection systems can utilize vein insertion detection sensors to determine when the needle has pierced the vein based on a back flow of blood into the needle. For example, an optical sensor can detect the presence of blood in a clear tube operably connected to the needle or in the lumen of the needle through a transparent needle housing. In a number of embodiments, pressure sensors can be used to detect a change in an operably connected tube when blood has entered the tube.

In some embodiments, vein insertion detection sensors can use electrochemical impedance to detect when an electrically conductive needle is in contact with the blood in a vein. In order to provide this kind of detection, needles in accordance with various embodiments of the invention can include one or more electrochemical electrodes that can be used for sensing the electrochemical impedance. Vein insertion detection in accordance with various embodiments of the invention can measure impedance at multiple zones of a needle. For instance, in a variety of embodiments, the needle can be first coated with an electrically insulating layer and then two electrically conducting electrodes can be created on the outside of the surface. When these electrodes come in contact with human tissue, one will be able to measure an impedance between the electrodes. As the needle penetrates different layers of tissue, one would expect changes in the impedance. However, once the vein is pierced, the conductivity of blood is significantly different than the surrounding tissue and so one would be able to unambiguously detect the presence of the blood and to stop moving the needle. Particularly in the case of vertical injections, needles in accordance with numerous embodiments of the invention can include sensors to detect the presence of blood both above and below a lumen opening to ensure that the lumen opening is contained within a vein. If the situation changes it could indicate an unexpected retraction event or motion of the vein. In a variety of embodiments, electrical connections can be built into the coupling assembly. Rather than an electrically insulating attachment, the attachment could have metal pads that when the needle assembly is properly inserted allows the electrical measurement to be made without having to connect additional wires.

Vein insertion detection sensors in accordance with a number of embodiments of the invention can include ultrasound to detect the different layers of the tissue ahead of the needle and determine when the needle is in direct contact with fluid. In particular, a similar needle design to that above could be used where in this case, the electrodes are configured to propagate an electromagnetic signal, or the needle itself could be vibrated to provide an ultrasonic signal. In a variety of embodiments, by using multiple types of vein insertion detection sensors, automatic injection devices can increase the accuracy of force measurements and of vein insertion detection. This method may be used as a kind of two-factor authentication for vein penetration and as an improved sensor, working with the disclosed force sensing mechanisms to more accurately determine when a vein has been punctured, subsequently stopping the injection and holding the needle in place.

### Additional Sensors

Sensors in accordance with numerous embodiments of the invention can include needle detection sensors that can be used to indicate the presence of a needle in the clamp mount. This may be, e.g., a photodetector that indicates the presence an object in or near the clamp mount, a pressure sensor to detect when something has been sufficiently locked into place, etc. Control systems in accordance with a number of embodiments of the invention can receive information from this sensor, and make an appropriate determination as to the presence of the needle. In some embodiments, no other action, or only certain actions, are allowed when a needle is not present, and other actions are restricted (such as lowering the clamp mount, etc.). In some cases, the system contains a microphone, allowing the system to be voice-activated and voice-controlled.

Although many of the examples described herein describe the use of a single sensor, one skilled in the art will recognize that similar systems and methods can be used in a variety of applications, including (but not limited to) the combination of signals from different sensors and/or the supplementing of a first sensor reading with readings from a second sensor, without departing from this invention.

### Needle Element

In numerous embodiments, needle elements may be a stand-alone needle, a needle attached to a hub, a needle on a syringe, and/or any other arrangement in which a needle can be provided. In some embodiments, needle elements may include a commercially available needle, such as a 32-gauge 4 mm needle, a 27-gauge 12.7 mm needle, a 26-gauge 12.7 mm needle, or a 25-gauge 15.9 mm needle. In certain embodiments, needle elements can be operably connected to a flexible tube, which is adapted to transport a liquid medication to the needle for injection, the flexible tube being operably connected to, e.g., a syringe or pump that pressurizes medication for transport to a subject. However, current needles meant for intravenous delivery or extraction of fluids are designed for an injection that would be performed at a shallow angle relative to the vein. The disclosed technology is different in that it is specifically designed for the injection to be performed vertically, coming down at a 90-degree angle perpendicular to the skin and vein. Other needle designs on the market are designed for vertical intramuscular injections, rather than being designed for vertical angle into a vein.

Needle elements in accordance with numerous embodiments of the invention can be custom needle assemblies used to position and inject a needle, and may include various components for a needle, such as (but not limited to) a coupling assembly, a needle housing, and a needle.

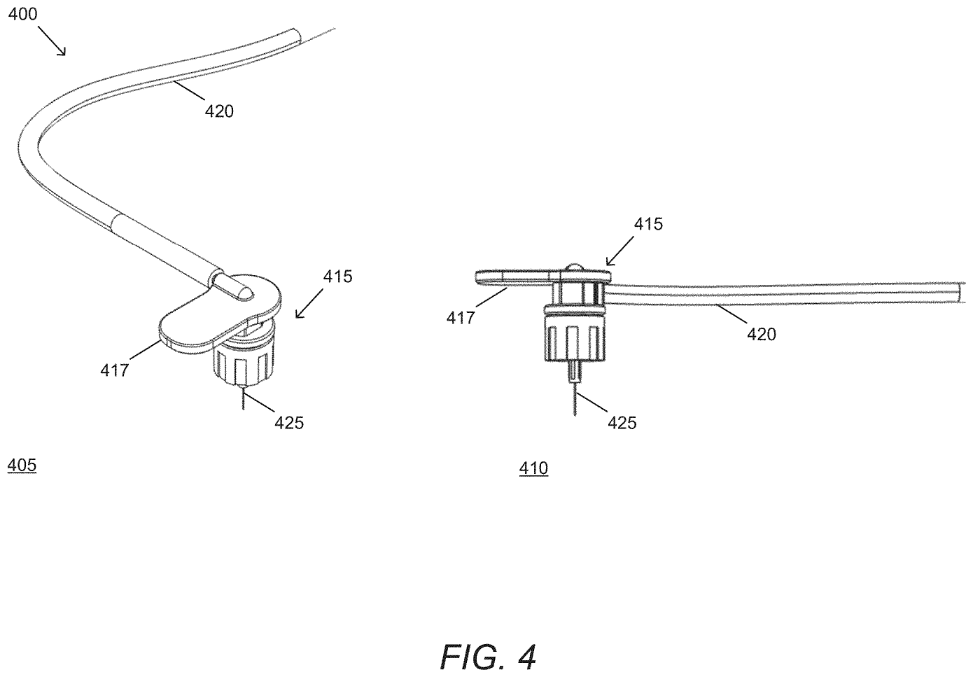

An example of a needle element in accordance with an embodiment of the invention is illustrated in FIG. 4 in two views 405 and 410. Needle element 400 includes coupling assembly 415, tubing 420, and needle 425. Coupling assemblies in accordance with several embodiments of the invention can be used to couple the needle to an automatic injection device and/or to other elements (e.g., tubing 410, a blood drawing apparatus, a syringe, etc.). Coupling assembly 415 includes a tab 417 for inserting the needle element into an injection compartment. Coupling assembly 415 also attaches to tubing 420 perpendicular to the needle 425. Materials that are drawn or injected through the needle can pass through the coupling assembly. Needle element 400 does not include a needle housing.

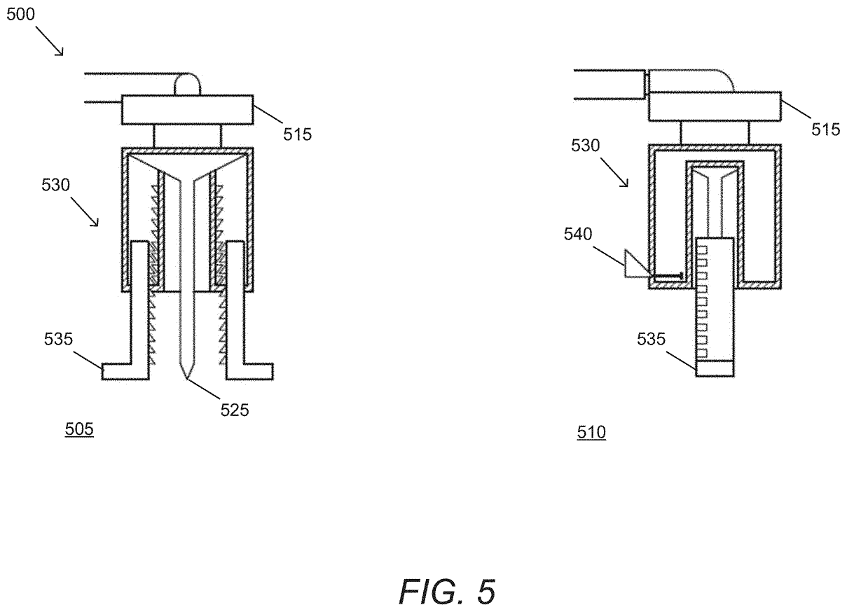

An example of a needle element with a needle housing in accordance with an embodiment of the invention is illustrated in FIG. 5 with a front view 505 and side view 510. In this example, needle element 500 includes coupling assembly 515, needle 525, and needle housing 530. Needle housing 530 includes retractable support elements 535 and locking mechanism 540. Needle housings in accordance with a number of embodiments of the invention can perform various functions, such as (but not limited to) stabilizing a needle for injection, providing a sterile injection point, and/or limiting a needle's penetration into the skin.

Examples of various components of needle elements in accordance with many embodiments of the invention are described in greater detail below. Needle elements in accordance with some embodiments of the invention can be configured for easy installation and removal from an automatic injection device.

### Coupling Assembly

Coupling assemblies in accordance with numerous embodiments of the invention can be used to secure needle elements within an automatic injection device (e.g., to a securing mechanism). In numerous embodiments, coupling assemblies can include a tab for easy installation of a needle element. Coupling assemblies in accordance with some embodiments of the invention can utilize various ways to couple with an automatic injection device, including (but not limited to) clamp mounts, clips, screws, bolts, adhesive, hook and loop tape, snap fit, friction fit, magnets, etc.

In many embodiments, coupling assemblies can include a connection between a lumen of the needle and other external elements (e.g., pumps, sample vials, syringes, etc.). Connections for coupling assemblies in accordance with certain embodiments of the invention can be made perpendicular to the orientation of the needle, allowing for a reduced height for the housing of an automatic injection device. When injections have a rotating component, coupling assemblies in accordance with certain such embodiments of the invention can provide a rotating element to allow for 360 degrees of rotation of the needle.

Coupling assemblies in accordance with a number of embodiments of the invention can assist in the sensing aspects of an automatic injection device. In a number of embodiments, the top of the coupling assembly can be optically transparent, allowing for a light source and/or detector to be positioned above the needle element to pass light (e.g., LED, laser diode, etc.) through the lumen of the needle and/or to monitor an optical signal through the lumen. For example, this could be used to detect the first flash of blood in the needle, thereby confirming that the needle is properly inserted in the vein. In various embodiments, such sensors can be used to ensure that the needle remains properly inserted in the vein. In some embodiments, blood passing through coupling assemblies can be analyzed to monitor various measures, such as (but not limited to) oxygenation levels, glucose levels, and/or other medical indicators of value to medical professionals. In a number of embodiments, such information can be used to inform the injection process and/or reported back to the control app over Bluetooth or other connectivity.

In the case where the tip of the needle is hollow, this coupling assembly can be used to detect the location of the veins on the surface. IR/Red light can be transmitted through the needle and into the skin below the needle prior to insertion. The corresponding detector can, based on the absorption, reflectance, or fluorescence determine whether a vein is below the needle or not. Using such a method to detect the location of a vein will ensure improved accuracy as compared to imaging or scanning the arm and then mechanically returning to a given place.

### Needle Housing

Needle housings in accordance with certain embodiments of the invention can be used to stabilize an injection site, protect a needle from being damaged, prevent a needle from going too deep, and/or sterilize an injection site. Needle housings in accordance with many embodiments of the invention can provide protection for the needle preventing bending or breaking during loading. In numerous embodiments, needle housings can be designed to prevent the occurrence of a needle being vertically injected slipping too far vertically and puncturing the bottom wall of a vein instead of just the top wall. Instead of having the needle housing serve the sole purpose of connecting to tubing or syringes, needle housings in accordance with numerous embodiments of the invention can act as a stabilizer that rests on the patient's skin and takes the bulk of unnecessary downward force or pressure after the needle is successfully in the vein. In various embodiments, needle housings can extend down towards or beyond the needle itself to rest on the skin of the user to provide a point of support to diffuse downward pressure onto the skin of the user. Otherwise, the slightest bit of unwanted downward pressure (someone bumps into the user, they cough, etc.) could cause the needle to push down into the user's skin further than necessary and puncture the bottom of the users vein, causing unwanted pain and medical complications.

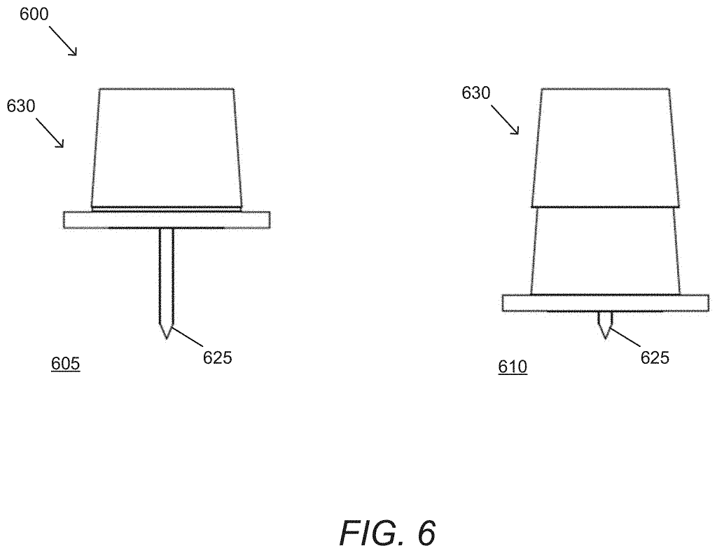

An example of a needle housing is described above with reference to FIGS. 5, where the housing includes two legs that extend down toward the injection surface and can be locked into place once the vein has been entered. Another example of a needle housing is illustrated in FIG. 6. The first view 605 illustrates that the needle housing 630 is retracted, exposing a length of the needle 625. Needle housings in accordance with a number of embodiments of the invention may be locked in a retracted position once a vein has been entered. In the second view 610, needle housing 630 is extended to completely surround needle 625. Needle housing 630 is mostly extended so that only the tip of needle 625 is visible.

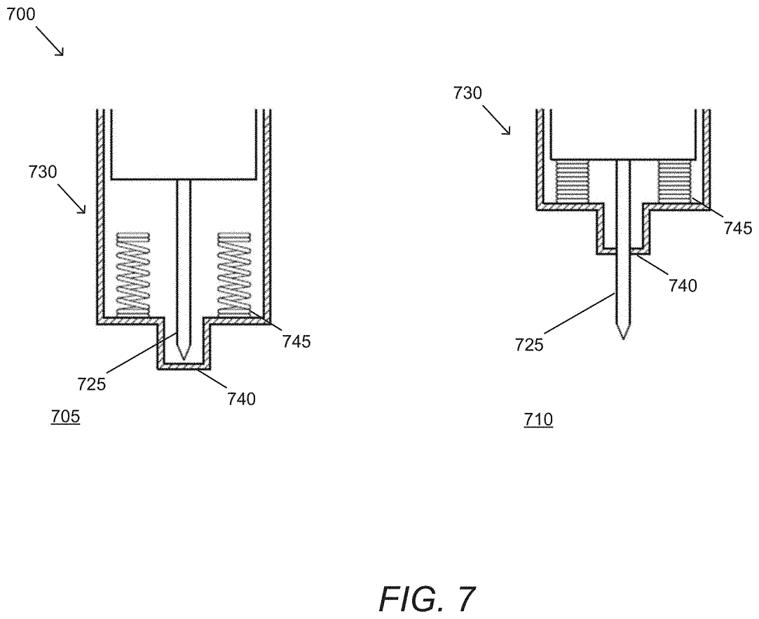

An example of a retractable needle housing in accordance with an embodiment of the invention is illustrated in FIG. 7. In this example, needle housing 730 covers needle 725. Needle housing 730 includes an opening 740 and springs 745. The first view 705 shows needle housing 730 in a retracted position, where the needle 725 is not exposed. In some embodiments, needle housing openings can be covered (e.g., in a polymer, metal, etc.), protecting the needle from exposure until the injection process begins. The second view 710 illustrates that the needle has been injected and the tip of the needle 725 is exposed. Springs, or other retraction mechanisms, can be used with needle housings in accordance with many embodiments of the invention to retract the needle or to otherwise sheathe the needle (e.g., after the injection process, when the device is accidentally removed, etc.). In a variety of embodiments, needle housings can be locked in an exposed position with a locking mechanism during the injection process. In many embodiments, when the needle housings are unlocked after an injection, they may automatically be retracted into the needle housing. Although specific examples of needle housings are illustrated in FIGS. 5-7, any of a variety of needle housings can be utilized similar to those described herein as appropriate to the requirements of specific applications in accordance with embodiments of the invention.

In several embodiments, needle housings can include a retraction mechanism that allows the housing to retract when it comes in contact with the surface of the skin, while the needle continues to penetrate into the skin. Needle housings in accordance with many embodiments of the invention can also include a locking mechanism that prevents the needle housing from retracting any further once it has been determined that a needle has entered a vein. Once the vein is accessed and the mechanism is locked in place, the needle and attachment are now acting as one unit, so now a unforeseen forceful pressure on the needle no longer places all of the stress on the needle itself, but instead on the attachment which displaces the pressure on the surface of the skin, alleviating the risk of the needle getting pushed further into, and potentially through, the vein.

This protective housing may extend beyond the tip of the needle(s), leaving an opening for the needle to pass through during the injection in accordance with various embodiments of the invention. In certain embodiments, the opening of the housing can be at least the diameter of the widest part of the needle. Needle housing openings in accordance with many embodiments of the invention may be no more than twice the diameter of the needle in order to prevent the needle from possibly bending prior to insertion.

In a variety of embodiments, needle housing openings may be covered with a protective polymer sheath without a hole in it. As the sheath needle housing makes contact with the skin, the needle can first penetrate the sheath and then the skin. This can prevent damage to the needle, as well as maintain the needle in a sterile environment prior to injection.

The tip (or bottom) of the needle housing in accordance with certain embodiments of the invention can be flat or it could have other shapes. For instance, having a sawtooth design of one or more triangular protrusions would assist in keeping the needle housing in fixed contact with the skin. The protrusions should be small enough to not cause damage to the skin, but large enough to provide good friction to the surface. Similarly the tip of the housing could have gentle (e.g. curved) but larger protrusions that would assist vein-roll prevention as described herein.

Needle housings in accordance with a number of embodiments of the invention can include a retraction mechanism for withdrawing a needle after an injection and/or when an injection has failed. Retraction mechanisms in accordance with various embodiments of the invention can be spring loaded and/or friction attached so that friction keeps the housing in place and once the housing is retracted, it remains in that state. In several embodiments, a spring is fit over the needle (e.g. the needle passes through the center of the spring), where one side of the spring is in contact with the needle connector and the other side is in contact with the protective housing. When the protective housing comes in contact with the skin, the spring compresses moving the bottom of the housing closer to the bottom of the connector. In a number of embodiments, rather than a single spring, multiple springs can be used in the housing to ensure smooth operation during. In a number of embodiments, needle housing tops can be designed with one or more longitudinal grooves, where the needle housing can have a corresponding tab that fits into the groove. In this way, the needle housing can remain in the proper orientation to ensure safe and easy retraction.

In some embodiments, the top of the needle housing can include a built-in force sensing apparatus that can detect the force that is pushing the needle into the skin/vein. In one embodiment, the force sensing apparatus is a microscale load cell which is electrically connected to one or more processors in order to measure the force/displacement of the needle as it penetrates the vein. In a variety of embodiments, such force measurements can be provided to a control system to determine whether a vein has been entered. In various embodiments, the top of the needle housing can include a connecting mechanism, such as (but not limited to) circular threading, female LUER-LOCKs, etc. Connecting mechanisms can be used to connect the needle to other elements (e.g., to the male LUER-LOCKs of syringes or tubing).

In several embodiments, needle housings can provide a sterile microenvironment that reduces the risk of outside environmental factors interfering with the sterility of the injection is provided. The sterilized environment is the result of a small micro-environment that is created within the VIT system. The key is that this small open space that surrounds the needle and the needle connector of the VIT system will not be a closed space, reducing the risk of outside contaminants throughout the duration of the injection. In numerous embodiments, needle housings can employ a suction mechanism that lowers the air pressure within this environment, allowing everything to stabilize. This suction can help reduce vein rolling as well. In some embodiments, once the sterile chamber has been placed onto the skin, the area in which a needle is strapped into a VIT system will be completely closed off via suction mechanisms to create a sterilized environment that reduces the risk of outside environmental factors interfering with the sterility of the injection. In other embodiments, or at other times, a positive pressure environment may be generated by providing additional “clean” air to the micro-environment. One approach to providing the small micro-environment is by constructing a small sterile chamber in which the needle can be positioned, and the air pressure controlled as needed. In some embodiments, the sterile chamber contains a small port through which air can be added or removed from the chamber.

In some embodiments, prior to moving the needle vertically into the vein, the small sterile chamber can be moved above the skin, and the area can be flushed with alcohol or other sterilization media prior to injecting the needle. The chamber would remain in place during the injection and a second flush of sterilization media can be used prior to removing the needle to further protect from infection. In some embodiments, a port in the sterile chamber is used to allow the sterilization media to be injected into and through the sterile chamber. In some embodiments, the sterilization media is sprayed into the chamber, onto or past the needle, and down onto the skin. In a number of embodiments, the small chamber can be connected directly to the needle, or it could be moved by a separate mechanism while attached to the system.

### Needle

Needles in accordance with numerous embodiments of the invention can include a shaft, a tip, and lumen openings. Needles for vertical injection in accordance with several embodiments of the invention can utilize various tips and/or lumen openings to minimize the discomfort caused to a patient, while also allowing for optimized force readings that can more clearly determine when a needle has entered a vein. When there is less friction or resistance force encountered when pushing the needle into the vein, the less force is required and therefore the smaller and more precise motors that can be used for automated injection devices. In addition, lower friction reduces the chance for discomfort as well as help needle remain on track. However, when friction is too small, changes in friction/force encountered when piercing a vein can be difficult to detect. Similarly, if the resistance is too big, then one would need a larger force and the result could be a large elastic recovery of the vein when the needle pierces the wall. This could cause the needle to overshoot through the vein.

In certain embodiments, the shaft of a needle is textured to emphasize force readings when a needle enters a vein. For example, the surface of a needle may include one or more protrusions that may cause distinct force readings when they enter into a vein. In many embodiments, multiple protrusions may be used, where the needle is configured to allow a smaller protrusion to enter the vein triggering a distinct force reading, while a second larger protrusion will not enter the vein because of the force required to push the protrusion through. This can help with vein entry as well as with keeping a needle within the vein.

In various embodiments, a sawtooth sidewall or roughened sidewall can be combined with an oscillating needle injection/extraction approach to act more like a saw when penetrating into the skin which would further reduce frictional forces. When using a sawtooth or other roughened pattern, automatic injection devices in accordance with a variety of embodiments of the invention can use vibration to extract the needle so that there is less discomfort.

In some embodiments, the needle is not a single needle, but a multipronged needle (having a primary needle and one or more secondary needles) designed to both inject and hold a vein in place. Secondary needles (typically needles located at some radial distance from a central, primary needle) in accordance with various embodiments of the invention can be optimized for diameter and shape so that they would be able to penetrate the skin, but not penetrate a vein when coming into contact. This can be achieved by controlling the curvature of the tip. A collection of smaller needles has the potential benefit to improve the volume of blood extracted or the delivery of medication while keeping the needle size small and the potential discomfort to the patient reduced.

In some embodiments, the needle is not a simple needle, but a needle and catheter combination for which once the needle pierces the vein, jt can be removed leaving a plastic or metal catheter behind in the vein for subsequent delivery of medication.

### Tip Shape

Needle tips in accordance with numerous embodiments of the invention can have various shapes based on the application. In the case of vertical injections, it can be desirable to have a symmetrically shaped tip. Traditional beveled needle tips designed for angled access may exert sideways forces when inserted vertically. In various embodiments, needle tips can come to a sharp point with an acute angle less than 40 degrees (measured from the tip to the sidewalls of the needle shaft) to reduce the force necessary to insert the needle and to minimize discomfort.

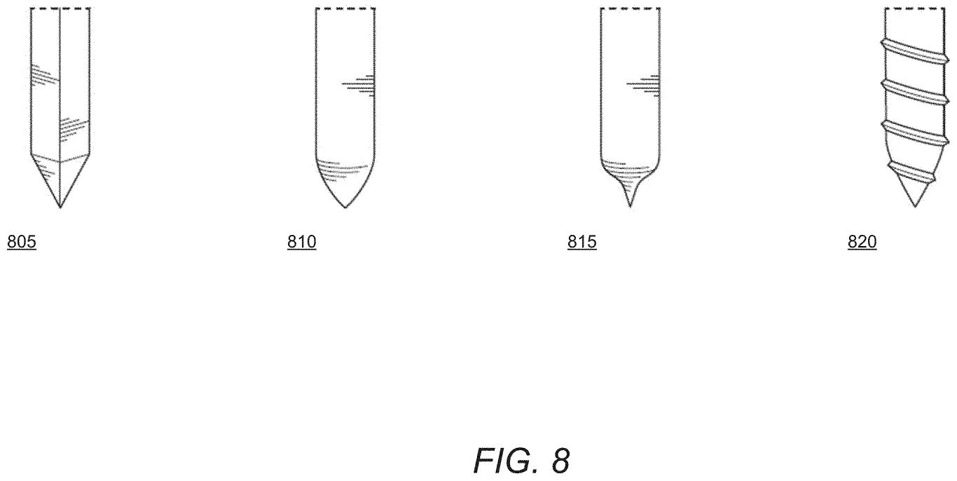

Examples of needle tips in accordance with some embodiments of the invention are illustrated in FIG. 8. This figure illustrates a triangular needle tip 805, a conical needle tip 810, a non-linear needle tip 815, and a screw needle tip 820. In numerous embodiments, the tip of the needle could be curved downward rather than tapered to a sharp point. By keeping the taper angle small, the friction along the sidewalls during injection could be minimized. Or by using an oscillatory motion and a rough walled needle the friction could be reduced. However, by curving the tip in accordance with some embodiments of the invention, it is possible that this would provide assistance against the possibility of vein rolling due to off center injections.

In a variety of embodiments, needle tips may have a non-linear taper. A conical tip has a linear taper from the shaft diameter to the point. However, this is not necessary and some potential benefit may be imparted by providing a taper that is either concave up or concave down. In the case of the automatic injection, non-linear tapers could allow for finer control of the friction/force required during the injection process. As a result one could further optimize the force measurement by created a taper design that provides a maximum difference once the needle pierces the vein.

In various embodiments, a screw type pattern on the outside surface of the needle would allow the needle to be injected through rotation into the skin similar to a screw or drill bit. This would further minimize the force required (benefit of the mechanical advantage provided by the screw pattern). In addition, it would guarantee that the needle does not slip out of the skin/vein during the injection process. A screw injection process could necessitate a different type of force measurement (torque versus force). Extraction of this needle would be accomplished by counter-rotating the needle.

In numerous embodiments, surface of the needle tips may not be smooth, but rather be textured (e.g., sawtoothed, ridged, etc.). The patterns could be regular or they could be random in accordance with various embodiments. Regular patterns could include sawtooths going along the longitudinal direction of the needle or circumferential rings, or a spiral (screw) pattern. The benefit of such a structure would be to assist in keeping the needle in the skin/vein and maintaining the direction during the injection process. Also, by having a roughened surface, a smaller surface area of the needle will be in contact with the tissue during insertion and thereby would have the ability to reduce the force required to inject a needle. Minimization of force would allow for smaller automatic injection devices by going to smaller motors and motion control systems. It could also provide greater accuracy and for higher resolution force sensors.

### Lumen Openings

Current IV needles use one big beveled hole that faces up on the beveled slant. This is because as a needle is injected laterally into a vein with its bevel facing up, the beveled opening is facing upwards in the vein instead of down towards the bottom wall of the vein. This allows for medication to then easily flow out (or blood to flow in) of the needle and into the vein without taking up too much space and blocking blood flow entirely. When injecting vertically, it may not be beneficial for the bevel to be arranged this way. If it is, and there is a needle sticking vertically in the middle of the vein, there is a chance that this needle will reduce the blood flow of the vein in the case of a small vein and a large needle. Since the diameter of most needles are not that much smaller than the diameter of a vein, if a solid needle is taking up the entire inside space of the vein, it might stop blood flow and cause issues. Needles in accordance with several embodiments of the invention may have one or more lumen openings around the needle tip in order to promote free blood flow. For example, in certain embodiments, needles can have a triangular needle tip with four sides, where each of the four sides has one or more holes to promote the free flow of liquids regardless of the presence of the needle tip. Such needles could take up the entire inside of the vein and blood would still flow freely through one bevel and out a bevel on the other side (like a pasta strainer). The lumen openings should be kept to the region of the needle that would be expected to be in the vein (add dimension of vein ˜1 mm or less) and the number of openings should be maximized in such a way to allow the maximum amount of blood flow while maintaining structural strength during the injection process.

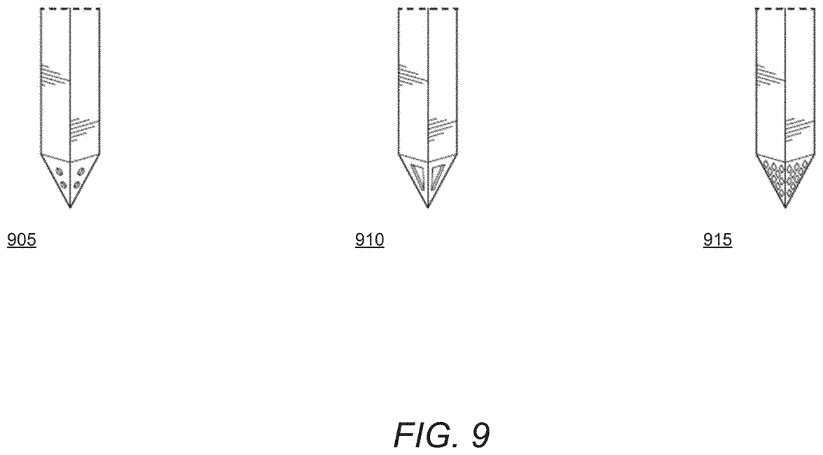

Examples of lumen openings in accordance with a number of embodiments of the invention are illustrated in FIG. 9. Specifically, this figure illustrates a needle tip with multiple lumen openings on each face 905, a needle tip with larger rectangular openings 910, and a latticed tip 915. Latticed tips in accordance with numerous embodiments of the invention can provide a structure whereby the remaining needle material forms an interlinked structure that is able to withstand compressive stress as well as shear stresses that occur during needle injection and extraction. In various embodiments, the openings may be circular, diamond, square, rectangular, or other shapes. In many embodiments, circular openings may provide a more optimal mechanical benefit as that would mean a lack of sharp corners that can lead to stress concentration and potential fracture of the needle. In various embodiments, lumen openings can be cut in a spiral pattern in the case of a needle designed to be injected through a rotation motion. This would provide the optimal mechanical strength against the shear forces acting during insertion/extraction. In a number of embodiments, lumen openings along the taper can be combined with textured surfaces to form a sawtooth pattern described above or create other roughness on the sides.

### Methods for Automatic Intravenous Injection

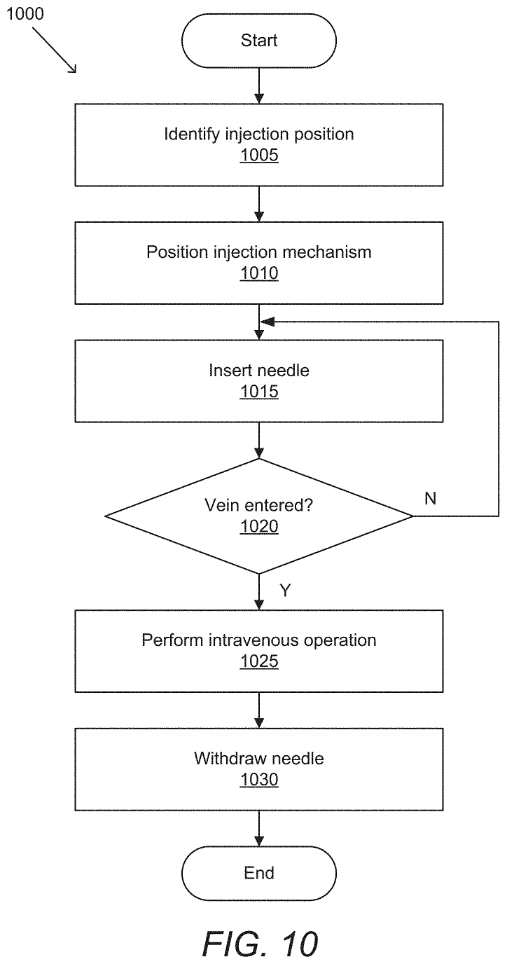

A process for automatic injections in accordance with an embodiment of the invention is illustrated in FIG. 10. Process 1000 identifies (1005) an injection position. Identified positions may be located on a plane parallel to the surface of the body. In a variety of embodiments, identifying an injection position can include generating a 2-D map of the injection area of a user's skin (e.g., within a housing opening) and identifying the injection position on the 2-D map. Processes in accordance with a number of embodiments of the invention can identify an injection position using light sent or sensed through the needle.

Process 1000 positions (1010) an injection mechanism based on the identified injection position. In some embodiments, the system is calibrated such that the processor can determine, based on an image captured by the vein identification and location sensors, how far, and in what direction, the needle must move. For example, in some embodiments, the sensor that captures an image is configured such that the center of the sensor image is the point directly under the needle. If each pixel represents a 10 μm distance on the surface of the skin, and a vein is detected 500 pixels above the center of the sensor image, the processor may be configured to determine that it must move the needle 5 mm (10 μm×500) forward from its current position. In a number of embodiments, processes can position the injection mechanism along a single axis (e.g., cross-wise across a user's arm or leg).

Process 1000 inserts (1015) a needle at the identified injection position. Inserting the needle in accordance with a variety of embodiments of the invention can include retracting a needle housing to expose a needle during the injection. In a number of embodiments, prior to inserting the needle, processes can sterilize and/or pressurize the injection site (e.g., an injection compartment and/or some micro-environment around the needle).

Process 1000 determines (1020) whether a vein has been entered by the needle. When the process determines the vein has not been entered, the process can return to step 1015 to continue inserting the needle. In a number of embodiments, when the needle has been inserted beyond a given threshold (e.g., time and/or distance) without finding the vein, the needle is retracted and the process ends.

When the process determines that a vein has been entered, process 1000 performs (1025) an intravenous operation. Intravenous operations can include (but are not limited to) drawing blood samples, injecting medications, etc. Once the intravenous operation is complete, process 1000 withdraws (1030) the needle and the process ends.

While specific processes for automatic injections are described above, any of a variety of processes can be utilized to automatically inject a needle as appropriate to the requirements of specific applications. In certain embodiments, steps may be executed or performed in any order or sequence not limited to the order and sequence shown and described. In a number of embodiments, some of the above steps may be executed or performed substantially simultaneously where appropriate or in parallel to reduce latency and processing times. In some embodiments, one or more of the above steps may be omitted.

### Systems for Automatic Injections

### Automatic Injection System

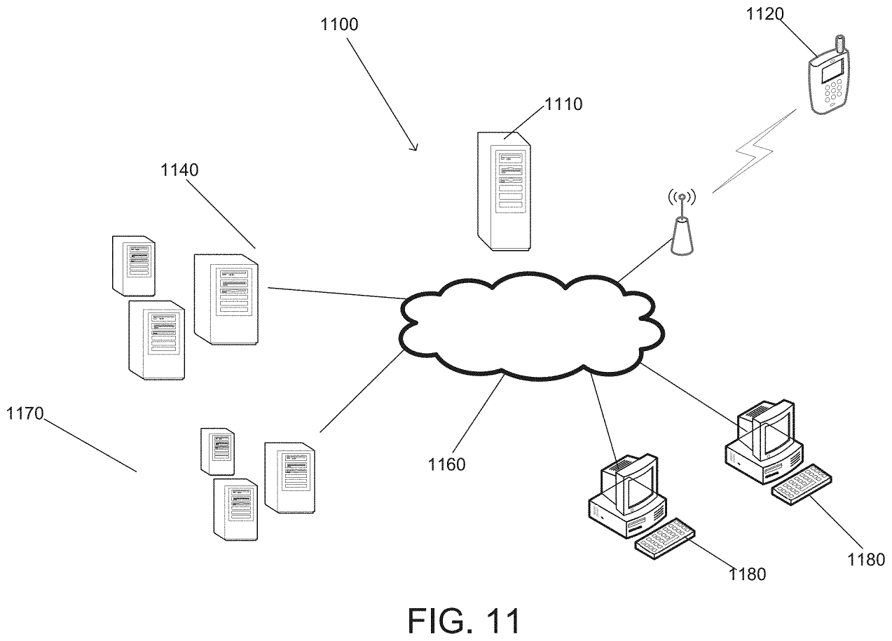

An example of an automatic injection system that automatically injects in accordance with an embodiment of the invention is illustrated in FIG. 11. Network 1100 includes a communications network 1160. The communications network 1160 is a network such as the Internet that allows devices connected to the network 1160 to communicate with other connected devices. Server systems 1110, 1140, and 1170 are connected to the network 1160. Each of the server systems 1110, 1140, and 1170 is a group of one or more servers communicatively connected to one another via internal networks that execute processes that provide cloud services to users over the network 1160. One skilled in the art will recognize that an automatic injection system may exclude certain components and/or include other components that are omitted for brevity without departing from this invention.

For purposes of this discussion, cloud services are one or more applications that are executed by one or more server systems to provide data and/or executable applications to devices over a network. The server systems 1110, 1140, and 1170 are shown each having three servers in the internal network. However, the server systems 1110, 1140 and 1170 may include any number of servers and any additional number of server systems may be connected to the network 1160 to provide cloud services. In accordance with various embodiments of this invention, an automatic injection system that uses systems and methods that automatically inject a needle and/or analyze information generated from an injection in accordance with an embodiment of the invention may be provided by a process being executed on a single server system and/or a group of server systems communicating over network 1160.

Users may use personal devices 1180 and 1120 that connect to the network 1160 to perform processes that automatically inject a needle in accordance with various embodiments of the invention. In the shown embodiment, the personal devices 1180 are shown as desktop computers that are connected via a conventional “wired” connection to the network 1160. However, the personal device 1180 may be a desktop computer, a laptop computer, a smart television, an entertainment gaming console, or any other device that connects to the network 1160 via a “wired” connection. The mobile device 1120 connects to network 1160 using a wireless connection. A wireless connection is a connection that uses Radio Frequency (RF) signals, Infrared signals, or any other form of wireless signaling to connect to the network 1160. In the example of this figure, the mobile device 1120 is a mobile telephone. However, mobile device 1120 may be a mobile phone, Personal Digital Assistant (PDA), a tablet, a smartphone, or any other type of device that connects to network 1160 via wireless connection without departing from this invention.

As can readily be appreciated the specific computing system used to automatically inject a needle is largely dependent upon the requirements of a given application and should not be considered as limited to any specific computing system(s) implementation.

### Automatic Injection Element

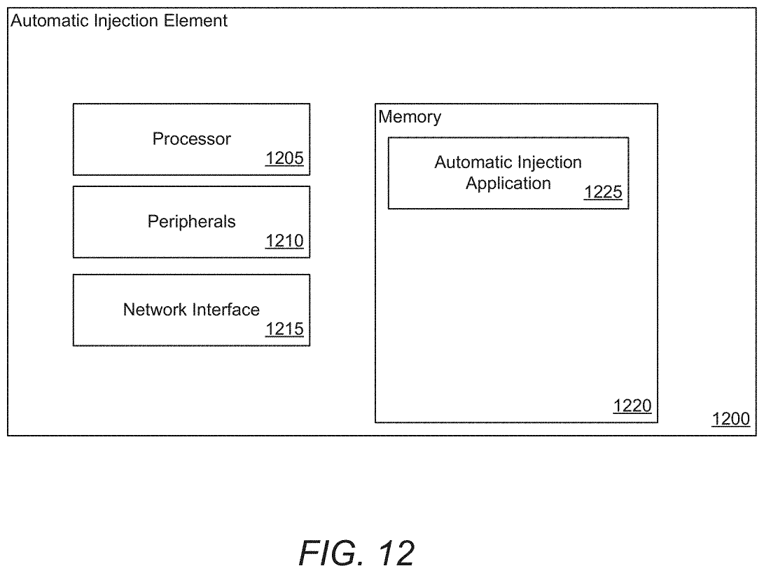

An example of an automatic injection element that executes instructions to perform processes that automatically inject a needle in accordance with an embodiment of the invention is illustrated in FIG. 12. Automatic injection elements in accordance with many embodiments of the invention can include (but are not limited to) one or more of mobile devices, cuffed devices, stationary injection devices, and/or other devices configured for automatic injections. Automatic injection element 1200 includes processor 1205, peripherals 1210, network interface 1215, and memory 1220. One skilled in the art will recognize that an automatic injection element may exclude certain components and/or include other components that are omitted for brevity without departing from this invention.

The processor 1205 can include (but is not limited to) a processor, microprocessor, controller, or a combination of processors, microprocessor, and/or controllers that performs instructions stored in the memory 1220 to manipulate data stored in the memory. Processor instructions can configure the processor 1205 to perform processes in accordance with certain embodiments of the invention.

Peripherals 1210 can include any of a variety of components for capturing data, such as (but not limited to) cameras, lights, lasers, displays, and/or sensors. In a variety of embodiments, peripherals can be used to gather inputs and/or provide outputs. Outputs in accordance with numerous embodiments of the invention can include instructions, notifications, and/or alerts. Automatic injection element 1200 can utilize network interface 1215 to transmit and receive data over a network based upon the instructions performed by processor 1205. Peripherals and/or network interfaces in accordance with many embodiments of the invention can be used to gather inputs that can be used to automatically inject a needle.

Memory 1220 includes an automatic injection application 1225. Automatic injection applications in accordance with several embodiments of the invention can be used to perform processes for automatically injecting a needle as described throughout this specification. Automatic injection elements in accordance with a number of embodiments of the invention may include any of a variety of combinations of components as described throughout this specification.

Although a specific example of an automatic injection element 1200 is illustrated in this figure, any of a variety of automatic injection elements can be utilized to perform processes for automatic injections similar to those described herein as appropriate to the requirements of specific applications in accordance with embodiments of the invention.

Although specific methods of automatic injections are discussed above, many different methods of automatic injections can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

1. A method for automatically injecting a needle into a vein, the method comprising:
identifying an injection position using a first set of one or more sensors;
positioning an injection mechanism at the identified injection position; and
vertically inserting a needle until entry in a vein is detected using a second set of one or more sensors.

2. The method of claim 1, wherein the first set of sensors comprises at least one of the group consisting of an infrared sensor, an optical sensor, and ultrasound.

3. The method of claim 1, wherein positioning the injection mechanism comprises moving the injection mechanism along a first axis and vertically inserting the needle comprises moving the injection mechanism along a second perpendicular axis.

4. The method of claim 1 further comprising securing a depth of the injection mechanism when entry in the vein is detected.

5. The method of claim 4, wherein:
the needle is part of a needle element,
the needle element comprises a needle housing, and
securing the depth of the injection mechanism comprises locking a position of the needle housing.

6. The method of claim 1 further comprising positioning a set of one or more vein roll prevention bars around the injection position.

7. The method of claim 1, wherein:
the second set of sensors comprises at least one of the group consisting of a force sensor, an electrochemical impedance sensor, a light sensor, and an ultrasound sensor;
the second set of sensors comprises first and second electrochemical impedance sensors;
detecting entry in the vein comprises detecting a presence of a liquid at both the first and second electrochemical impedance sensors; and
a set of lumen holes of the needle are located between the first and second electrochemical impedance sensors.

8-9. (canceled)

10. The method of claim 1 further comprising:
creating a pressurized space around the injection position; and
sterilizing the pressurized space around the injection position.

11. (canceled)

12. The method of claim 1, wherein vertically inserting a needle comprises:
vertically advancing a needle into a user, causing a needle housing surrounding the needle to retract; and
when entry in the vein is detected, locking the needle housing so that it cannot retract further.

13. The method of claim 1, wherein vertically inserting a needle comprises rotating the needle around an axis of the needle.

14. The method of claim 1, wherein vertically inserting a needle comprises oscillating the needle, wherein detecting entry in the vein comprises determining a force measurement based on an oscillation frequency of the oscillation.

15. (canceled)

16. The method of claim 1 further comprising generating a 2-D map using the first set of sensors and identifying the injection position based on the generated 2-D map.

17. An injection system for automatically injecting a needle, the injection system comprising:
a housing;
an injection mechanism comprising:
a securing element for securing a needle element;
a positioning element for positioning the needle element along a first axis; and
an insertion element for inserting the needle element along a perpendicular second axis; and
a set of one or more processors configured to:
identify an injection position using a first set of one or more sensors;
position an injection mechanism at the identified injection position along the first axis; and
vertically insert a needle along the second axis until entry in a vein is detected using a second set of one or more sensors.

18. The injection system of claim 17, wherein:
the housing comprises an injection opening and at least two roll prevention bars placed in parallel along the injection opening.

19. The injection system of claim 17, wherein the needle element comprises a coupling assembly for connecting the needle to the injection mechanism and the needle, wherein the coupling assembly provides a connection for liquids passing through the needle, wherein the connection is perpendicular to the needle.

20. (canceled)

21. The injection system of claim 19, wherein the needle element further comprises a needle housing that surrounds the needle, wherein the needle housing comprises a locking mechanism, wherein the set of processors is further configured to lock the locking mechanism when a vein is detected.

22. (canceled)

23. The injection system of claim 21, wherein the needle housing is a retractable needle housing that comprises a sheath that covers an opening of the needle housing.

24. (canceled)

25. The injection system of claim 19, wherein the needle comprises a first electrochemical sensor below a lumen opening of the needle and a second electrochemical sensor above the lumen opening.

26. The injection system of claim 17, wherein the needle tip is a non-linear tip.

27-64. (canceled)
